(19) United States
(12) Patent Application Publication
(10) Pub. No.: US 2021/0398275 A1
(43) Pub. Date: Dec. 23, 2021

(54) ORAL HEALTH PREDICTION APPARATUS AND METHOD USING MACHINE LEARNING ALGORITHM

(71) Applicant: QTT CO., Busan (KR)

(72) Inventors: Tae Yeon GO, Busan (KR); Wan Ho JANG, Suwon-si, Gyeonggi-do (KR); Jong Gu HAN, Namyangju-si, Gyeonggi-do (KR); Hee Young YANG, Busan (KR); Jay You CHOI, Seongnam-si, Gyeonggi-do (KR)

(21) Appl. No.: 17/285,651

(22) PCT Filed: Oct. 16, 2019
(86) PCT No.: PCT/KR2019/013563

§ 371 Date: Apr. 15, 2021

(30) Foreign Application Priority Data
Oct. 16, 2018 (KR) 10-2018-0123420
Oct. 14, 2019 (KR) 10-2019-0127250
Oct. 14, 2019 (KR) 10-2019-0127251

(51) Int. Cl. G06T 7/00; G16H 50/30; G16H 15/00

(52) U.S. Cl. CPC G06T 7/0012; G16H 50/30; G06T 2207/30036; G06T 2207/20081; G06T 2207/20084; G16H 15/00

(57) ABSTRACT

The present invention relates to an oral health prediction apparatus and method using a machine learning algorithm, which, when a user uploads an oral photo, comprehensively analyzes whether the user has braces, a dental caries state, a prosthesis state, and the like through a photo analysis using the machine learning algorithm, to enable exact prediction of oral health of the user. When a user provides an oral image of the user through a network by using a user terminal and requests an oral health determination result, the present invention analyzes the oral image by using the machine learning algorithm to predict an oral health state, analyzes a dental caries state and a prosthesis state through the analysis of the oral image, predicts the oral health state on the basis of the analyzed dental caries state information or periodontitis state information and prosthesis state information, and provides oral health state prediction information to the user.

### TECHNICAL FIELD

The present invention relates to an oral health prediction using a machine learning algorithm, and more particularly, to an oral health prediction apparatus and a method using a machine learning algorithm to comprehensively analyze a presence/absence of correction, a periodontitis status, a dental caries status, a prosthesis status, and the like through a photograph analysis using the machine learning algorithm when a user uploads an oral photograph so as to enable exact prediction of oral health of the user and provide a periodontal disease report.

### BACKGROUND ART

Although highly preventive effects may be obtained through proper preventive treatment and steady care against oral diseases, a lot of patients having oral diseases still have a lack of awareness about oral prevention, for example, the patients are reluctant to receive a diagnosis from a doctor or request a treatment unless a problem occurs in oral health.

In addition, accurate examination and diagnosis results and objective numerical information therefrom other than a subjective opinion of a dentist or dental hygienist are required for the prediction of a patient's current oral status. However, there is no system that objectively outputs an oral health status.

Two major oral diseases signify a tooth decay (dental caries) and a periodontal disease. In particular, since the periodontal disease, which means periodontitis and gingivitis, has no pain and progresses chronically in an early stage and accordingly a detection is delayed, it is a very difficult to prevent the periodontal disease in advance.

Accordingly, various methods for diagnosing a status of the oral disease in advance, improving an oral health and preventing the oral disease based on the above diagnosis have been researched and proposed.

Korean Unexamined Patent Publication No. 10-2017-0050467 (published on May 11, 2017) (METHOD AND SERVER FOR PROVIDING USER-COSTOMIZED ORAL CARE SERVICE) discloses that user condition information including information on at least one of gender, age, periodontal disease, and goal setting information of a user, and treatment information in the affiliated medical institution of the user are inputted from a user terminal. In addition, information on oral care products is inputted from an oral care product affiliated store. Then, information on specific oral care products included in a specific category corresponding to the user's condition information and treatment information is provided to the user terminal, so that the customized oral care services are provided.

In addition, Korean Patent Registration No. 10-1868979 (registered on Jun. 12, 2018) (SYSTEM AND METHOD FOR MANAGING ORAL CARE USING DEEP LEARNING) discloses that a patient's oral condition is examined, reference data for calculating oral examination index including oral examination data of a patient, and information on an residential area and an age are converted into big data and analyzed, and then an influence of the reference data for calculating oral examination index on an oral health according to the patient's residence area and age is determined based on a current viewpoint. Then, the reference data for calculating oral examination index is updated by adding weight to the reference data for calculating oral examination index, so that the oral health can be managed.

In addition, Korean Patent Registration No. 10-1788030 (registered on Oct. 13, 2017) (SYSTEM AND METHOD FOR RISK DIAGNOSIS ON ORAL DISEASE AND ORAL CARE) discloses that user's personal information, user's oral hygiene behavior data and oral inquiry data, and oral examination data generated by the user visiting a specialized medical institution are collected. Information collected in the above manner is integrated and analyzed to evaluate the user's risk of oral disease, and personalized services are provided.

However, since the conventional oral disease caring systems as described above are configured to generate oral disease caring information by analyzing the user's personal information, the oral data and inquiry data, and the oral examination data generated through specialized medical institutions, real-time performance is lowered. In addition, since the methods do not analyze a current oral image of the user, the accuracy in the analysis of oral diseases is insufficient.

In addition, according to the conventional technologies, it is difficult for the user to recognize his or her periodontal disease status in real time.

### DISCLOSURE

### Technical Problem

Accordingly, the present invention is proposed to solve the above problems in the related art. An object of the present invention is provide an oral health prediction apparatus and a method using a machine learning algorithm to comprehensively analyze a presence/absence of correction, a periodontitis status, a dental caries status, a prosthesis status, and the like through a photograph analysis using the machine learning algorithm when a user uploads an oral photograph so as to enable exact prediction of oral health of the user and provide a periodontal disease report.

Another object of the present invention is to provide an oral health prediction apparatus and a method using a machine learning algorithm to allow a user to manage a periodontal status in real time and periodically through a platform, and automatically book a hospital according to the user's request, thereby improving convenience.

### Technical Solution

In order to achieve the above-mentioned objects, a first embodiment of the “oral health prediction apparatus using the machine learning algorithm” according to the present invention includes: a user terminal for providing a periodontal image, personal information, and inquiry data of a user, and requesting a periodontal disease report; and a periodontal disease management server that analyzes the periodontal image provided from the user terminal by using a deep learning to generate a periodontal disease report and transmits the generated periodontal disease report to the user terminal.

The periodontal disease management server automatically searches for a hospital that responds to symptoms in the periodontal disease report upon request of a hospital reservation from the user terminal, and automatically makes a reservation in conjunction with a plurality of hospital servers.

In addition, a second embodiment of the “oral health prediction apparatus using the machine learning algorithm” according to the present invention includes: a user terminal for providing an oral image of the user and requesting an oral health decision result; and an oral health prediction server that predicts an oral health status by analyzing an oral image provided from the user terminal through a machine learning algorithm.

The oral health prediction server analyzes a dental caries status or periodontitis status, and a prosthesis status by analyzing the oral photographs, and predicts the oral health status based on the analyzed dental caries status information, periodontitis status information, and prosthesis status information.

In addition, the “oral health prediction method using the machine learning algorithm” according to the present invention includes: (a) registering the oral image provided from the user terminal as an oral health prediction target, by an oral health prediction server that predicts an oral health status by analyzing an oral image provided from a user terminal through a machine learning algorithm; determining, by the oral health prediction server, a presence or absence of an oral photograph by learning the oral image through a convolutional neural network (CNN) algorithm; determining whether the image is corrected and whether a tooth is extracted by learning the oral image through the CNN algorithm, by the oral health prediction server when the oral photograph is present; (d) obtaining dental caries status information or periodontitis status information and prosthesis status information by analyzing correction status information and tooth extraction status information determined by the oral health prediction server through an object detection; and (e) determining, by the oral health prediction server, an oral health status by learning the correction status information, the extraction status information, the dental caries status information, and the prosthesis status information through an artificial neural network (ANN) algorithm.

In addition, the oral health prediction method using dental caries detection according to the present invention further includes:

(f) transmitting the oral health prediction information obtained through the determination in step (e) to the user terminal.

### Advantageous Effects

According to the present invention, when a user uploads an oral photograph, a presence/absence of correction, a periodontitis status, a dental caries status, a prosthesis status, and the like are comprehensively analyzed in real time, so that the user's oral health can be accurately predicted.

In addition, the predicted oral health status information is provided to the user in the form of a report to induce the user to recognize and manage an oral condition in real time, so that deterioration of the oral health can be prevented in advance.

In addition, a hospital is automatically booked according to the user's request for a hospital reservation, so that the convenience of the user's hospital can be improved.

### DESCRIPTION OF DRAWINGS

FIG. 1 is a configuration diagram of a first embodiment of an oral health prediction apparatus using a machine learning algorithm according to the present invention.

FIG. 2 is a configuration diagram of an embodiment of the information analysis device of FIG. 1.

FIG. 3 is a configuration diagram of an embodiment of the report generation device of FIG. 1.

FIG. 4 is a flowchart showing an oral health prediction process using a machine learning algorithm according to the present invention.

FIGS. 5a and 5b are exemplary configuration diagram classifying affected parts of periodontitis according to the present invention.

FIG. 6 is an exemplary diagram for diagnosing an image using the machine learning algorithm according to the present invention.

FIG. 7 is an exemplary diagram showing exacerbation stages of a periodontal disease.

FIG. 8 is an exemplary view showing 12 periodontal sites photographed using a smartphone.

FIGS. 9a and 9d are exemplary views of an inquiry response sheet applied to the present invention.

FIG. 10 is an exemplary view of an inquiry diagnosis according to the present invention.

FIG. 11 is an exemplary diagram of a CNN model that is an image diagnosis system applied to the present invention.

FIG. 12 is a configuration diagram of a second embodiment of the oral health prediction apparatus using a machine learning algorithm according to the present invention.

FIG. 13 is a configuration diagram of an embodiment of the oral health prediction server of FIG. 1.

FIG. 14 is a flow chart of a first embodiment showing an oral health prediction method using a machine learning algorithm according to the present invention.

FIG. 15 is an exemplary diagram of determining the presence or absence of an oral photograph by learning oral photographs through the CNN algorithm according to the present invention.

FIG. 16 is an exemplary diagram of determining the presence or absence of orthodontic treatment by learning oral photographs through the CNN algorithm according to the present invention.

FIG. 17 is an exemplary diagram of detecting an oral disease and a prosthesis by learning oral photographs through an object detection algorithm according to the present invention.

FIG. 18 is an exemplary diagram of determining an oral health status by learning the presence of correction information, the detected oral disease information and the detected prosthesis information through a DNN algorithm according to the present invention.

FIG. 19 is a flow chart of a second embodiment showing the oral health prediction method using the machine learning algorithm according to the present invention.

### BEST MODE

Mode for Invention

Hereinafter, an oral health prediction apparatus and a method using a machine learning algorithm according to a preferred embodiment of the present invention will be described with reference to the accompanying drawings.

The terms or words used in the present invention described below should not be construed as limited to a conventional or lexical meaning, and should be construed as the meanings and concepts based on the principle that “an inventor may define the concept of the term properly in order to describe the invention in the best way”.

Accordingly, the embodiments described in the specification and the configurations shown in the drawings are merely preferred embodiments according to the present invention, and may not represent all of the technical ideas of the present invention. Therefore, it will be understood that various equivalents and modifications may be substituted therefor at the time of filing of the present application.

FIG. 1 is a schematic configuration diagram of an oral health prediction apparatus using a machine learning algorithm according to a first preferred embodiment of the present invention.

The oral health prediction apparatus using the machine learning algorithm according to the present invention includes a user terminal 10 and a periodontal disease management server 20.

Although not shown in the drawing, the periodontal disease management server 20 may make hospital reservations in conjunction with a plurality of hospitals (dental hospitals).

The user terminal 10 is connected to the periodontal disease management server 20 on an online basis through a network, and serves to provide a periodontal image, personal information, and inquiry data of the user and request a periodontal disease report. It is preferable that the user recognizes a periodontal disease status through the periodontal disease report by using the user terminal 10 and then take follow-up measures. The above user terminal 10 is a terminal used by the user, and may be implemented with a mobile device such as a smartphone and a smart pad, and a personal computer and a notebook computer that are capable of accessing to Internet. In the present invention, it is assumed that the user terminal is implemented as a smart phone as the embodiment.

The network serves to interface data between the user terminal 10 and the periodontal disease management server 20 to each other. The above network may be implemented as a data network, a wired/wireless network, a mobile communication network, and a public telephone network.

In addition, the periodontal disease management server 20 serves to analyze the periodontal image provided from the user terminal 10 by using deep learning to generate a periodontal disease report (oral health prediction information) and transmit the periodontal disease report to the user terminal 10. The periodontal disease report is not a result report of diagnosing the user's periodontal disease, but is a status report so as to allow the user to take preventive measures or follow-up measures (such as visiting a hospital) in which the periodontal disease status is provided by analyzing the periodontal image.

It is preferable that the above periodontal disease management server 20 automatically searches for a hospital that responds to symptoms in the periodontal disease report upon request of a hospital reservation from the user terminal 10, and automatically makes reservations in conjunction with a plurality of hospital servers.

To this end, the periodontal disease management server 20 may include: an information analysis device 21 for extracting analysis data after learning a user periodontal image by deep learning; a report generation device 22 for generating a periodontal disease report by analyzing the analysis data extracted from the information analysis device 21 and inquiry information based on big data to transmit the generated periodontal disease report to the user terminal 10; and a hospital reservation device 23 for searching for a hospital corresponding to symptoms of the periodontal disease report to automatically make a reservation when a hospital reservation is requested through the user terminal 10, and transmitting hospital reservation information to the user terminal 10.

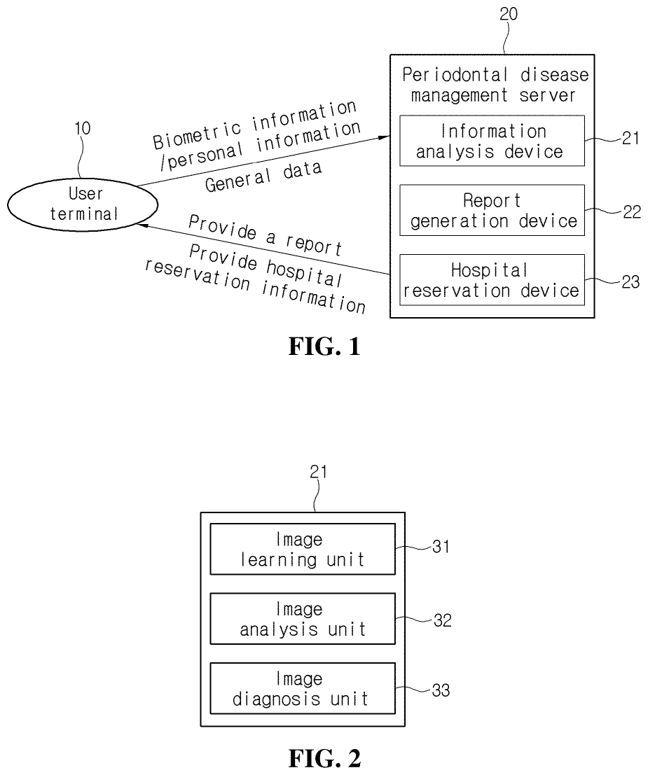

Preferably, as shown in FIG. 2, the information analysis device 21 includes an image learning unit 31 for learning a periodontal image, an image analysis unit 32 for analyzing results learned by the image learning unit 31, and an image diagnosis unit 33 for extracting periodontal disease analysis data by analyzing the periodontal image through deep learning based on the image analysis result.

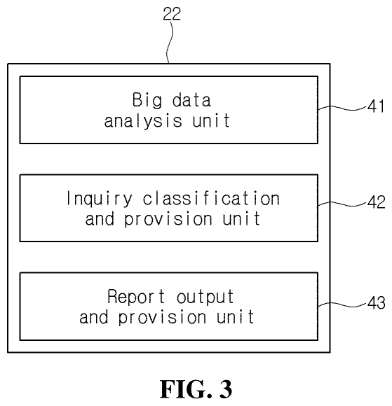

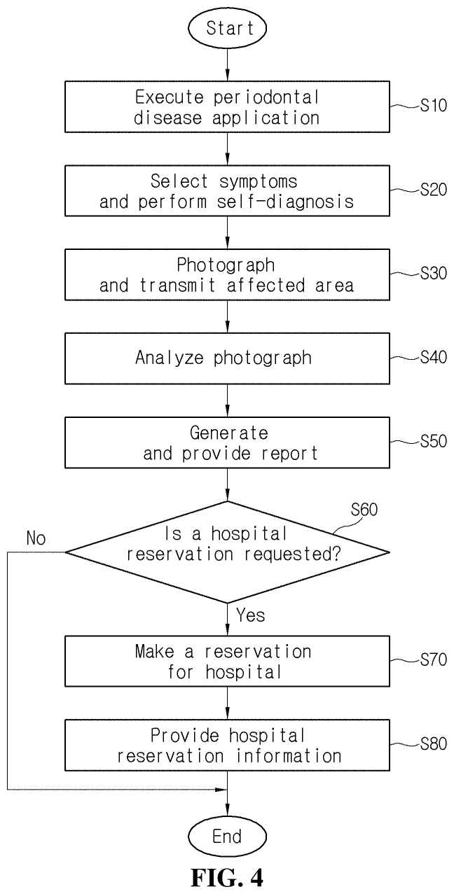

In addition, as shown in FIG. 3, the report generation device 22 includes a big data analysis unit 41 for analyzing periodontal disease analysis data provided by the information analysis device 21 based on periodontal disease big data, an inquiry classification and provision unit 42 for providing inquiry data to the user terminal 10, and classifying the inquiry information provided from the user terminal 10, and a report output and provision unit 43 for outputting a periodontal disease report based on the analysis result of the big data analysis unit 41 and the inquiry classification information of the inquiry classification and provision unit 42 and providing the outputted periodontal disease report.

In addition, the hospital reservation device 23 may preferably search and recommend a hospital having the shortest distance from the user's location based on a hospital name entered by the user or a self-recommended hospital and location information of hospitals around the user, and automatically make a hospital reservation in conjunction with a hospital server according to a hospital selection of the user.

More preferably, the hospital reservation device 23 may determine a ranking by evaluating hospitals using consumer evaluations and an independent hospital evaluation algorithm, search for a reservation hospital based on the ranking, and make a hospital reservation in conjunction with the hospital server according to a hospital selection the user.

Operations of a first embodiment of the oral health prediction apparatus using the machine learning algorithm configured in the above manner according to the present invention will be described in detail as follows.

First, the user downloads a periodontal disease application for checking and caring a periodontal disease status from the periodontal disease management server 20 and stores the periodontal disease application in the user terminal 10.

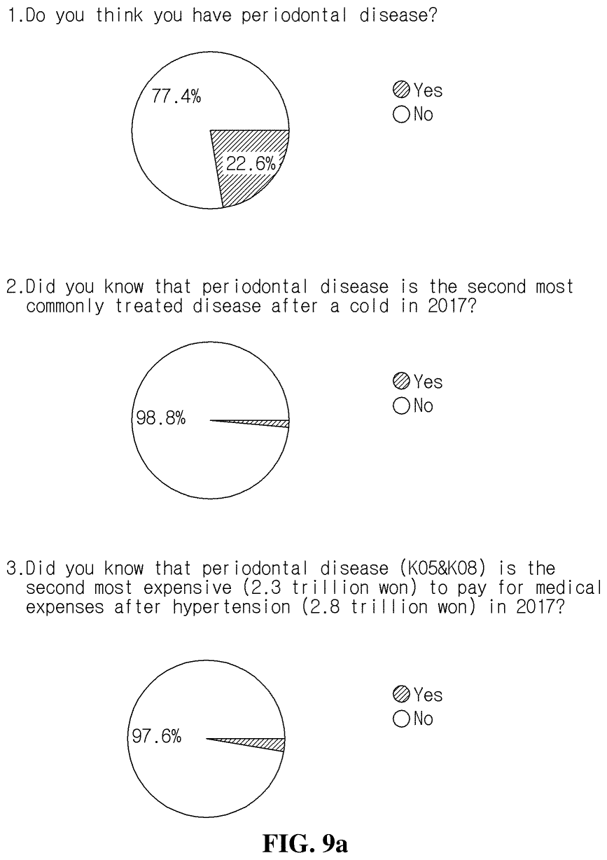

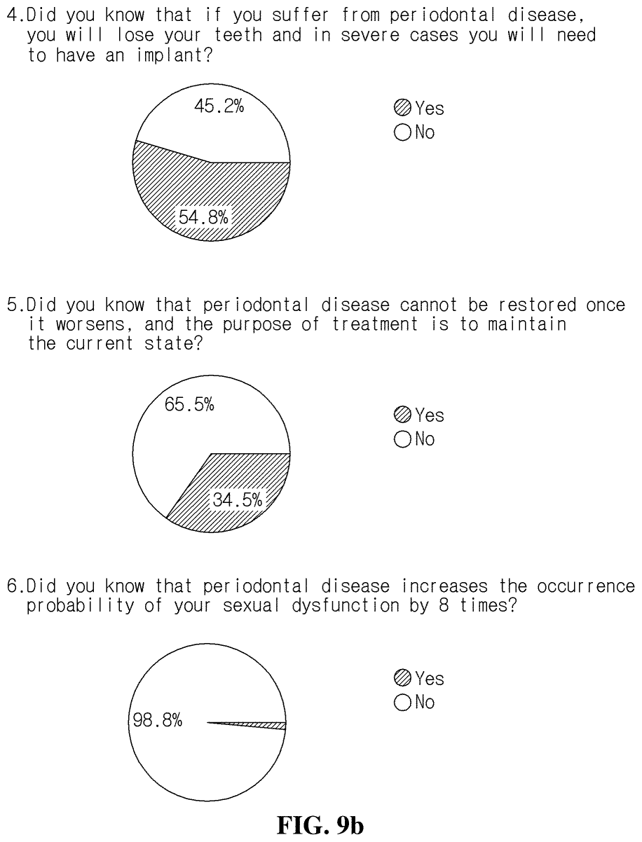

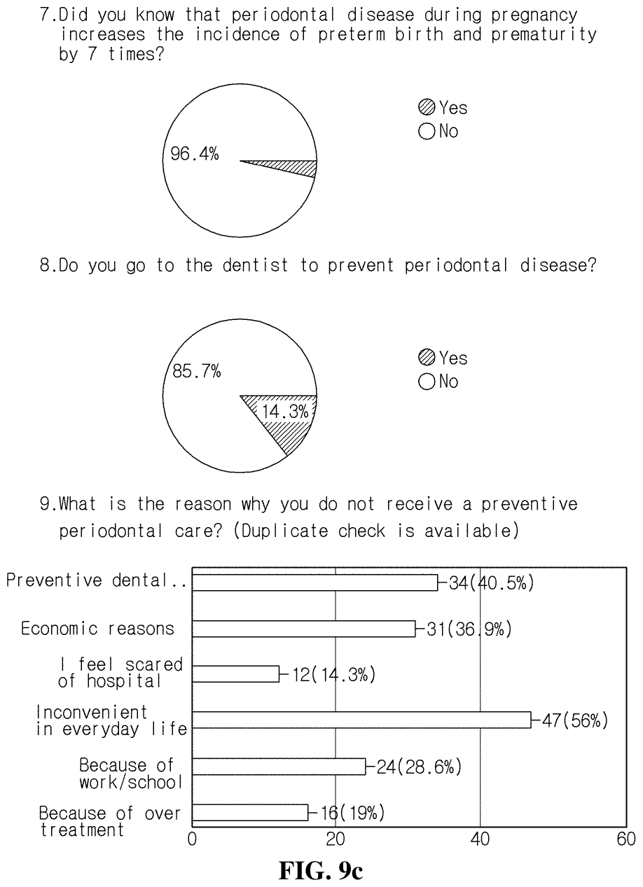

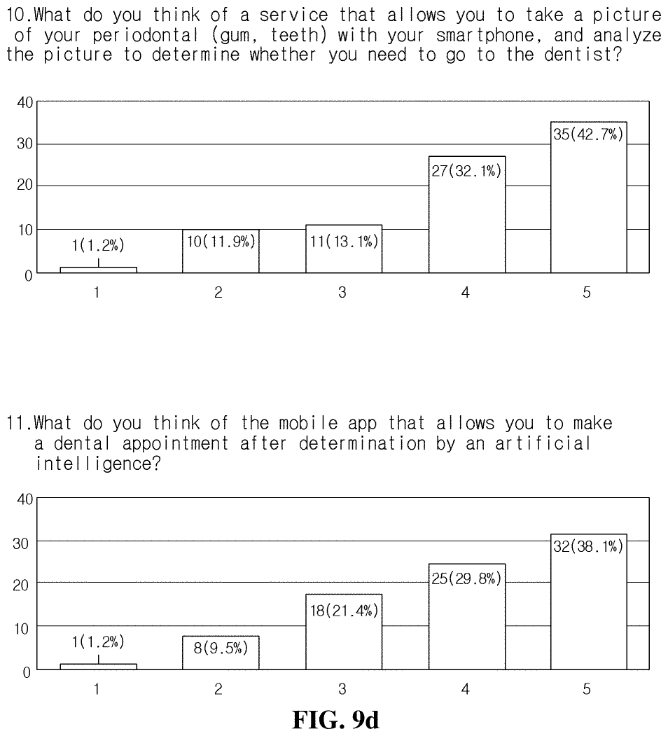

Then, in order to check the periodontal disease status, the user executes the periodontal disease application (S10), and tries performing self-diagnosis while selecting symptoms based on the inquiry data included in the periodontal disease application (S20). The inquiry data, as shown in FIGS. 9a to 9d, is developed in the form of a digital process based on diagnosis and treatment data and know-how accumulated for a long period of time by medical professionals who are experts in a dental general hospital, unlike general dental inquiries.

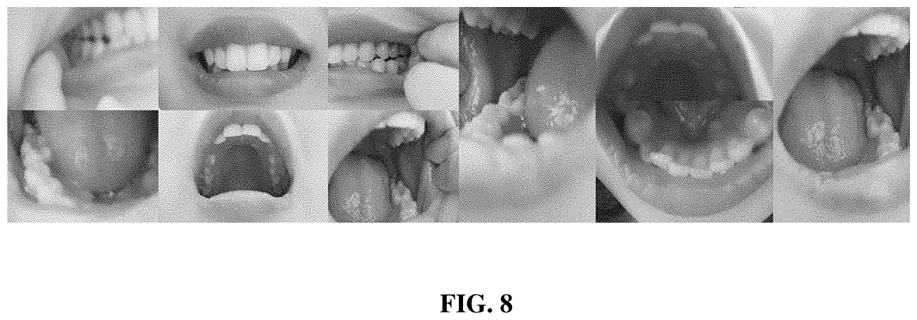

Next, when the user wants to check a personal periodontal disease status in real time, the user photographs a periodontal site to be checked for the periodontal disease status by using a camera provided in the user terminal 10, transmits the photographed periodontal image, the inquiry data information and personal information to the periodontal disease management server 20, and requests a periodontal disease report (S30). FIG. 8 shows exemplary photographs of 12 periodontal sites photographed by the user using a smartphone.

In the periodontal disease management server 20, when the periodontal image and the personal information and the inquiry data transmitted through the user terminal 10 are received, the information analysis device 21 extracts periodontal disease data by analyzing the periodontal image through an artificial intelligence (deep learning), and the report generation device 22 finally generates a periodontal disease status report through big data analysis based on the periodontal disease data and the inquiry information.

For example, since users personally and directly photograph and photographing environments are different, there is no standard for colors of actual photographed images. Accordingly, the information analysis device 21 automatically sets a reference point with respect to the transmitted periodontal image data, corrects colors of all photographs to correspond to the reference point, and then saves the corrected photographs. Then, the image learning unit 31 learns the received periodontal image, and the image analysis unit 32 analyzes the result learned by the image learning unit 31. Then, the image diagnosis unit 33 extracts periodontal disease analysis data by analyzing the periodontal image through deep learning based on the image analysis result. The periodontal disease analysis data refers to preliminary data for analyzing the periodontal disease using big data related to the periodontal disease.

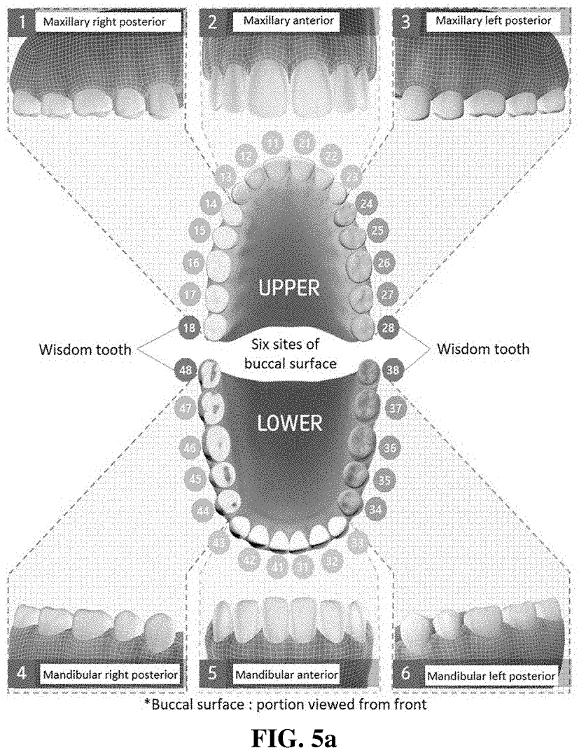

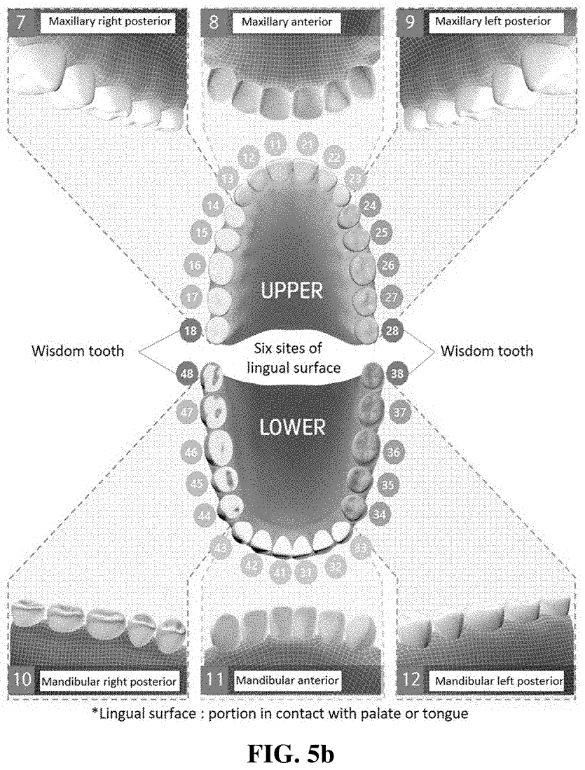

In other words, an accurate site corresponding to the image of the periodontal site presented by the user is checked, a position of the periodontal site image is confirmed, and then the image learning is performed. The periodontal disease is divided into periodontitis and gingivitis, and an affected area of periodontitis is generally classified into 12 sites for the analysis of the periodontitis as shown in FIG. 5. Therefore, the periodontal images transmitted by the user are compared to the classified 12 sites to check accurate affected areas. Then, the image analysis is performed based on the learning results.

When the image analysis is completed, the image is diagnosed through deep learning, and periodontal disease analysis data for periodontal disease analysis is extracted from the diagnosis result.

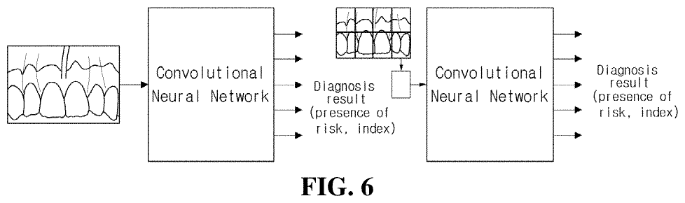

As shown in FIG. 6, the deep learning is trained through data outputted by medical professionals with clinical tests, not through the data-based learning that has been previously conducted. The periodontal images photographed by the patient at 12 the sites are divided into 8 sites again, and the images are analyzed more accurately through neural network analysis through CNN (S40).

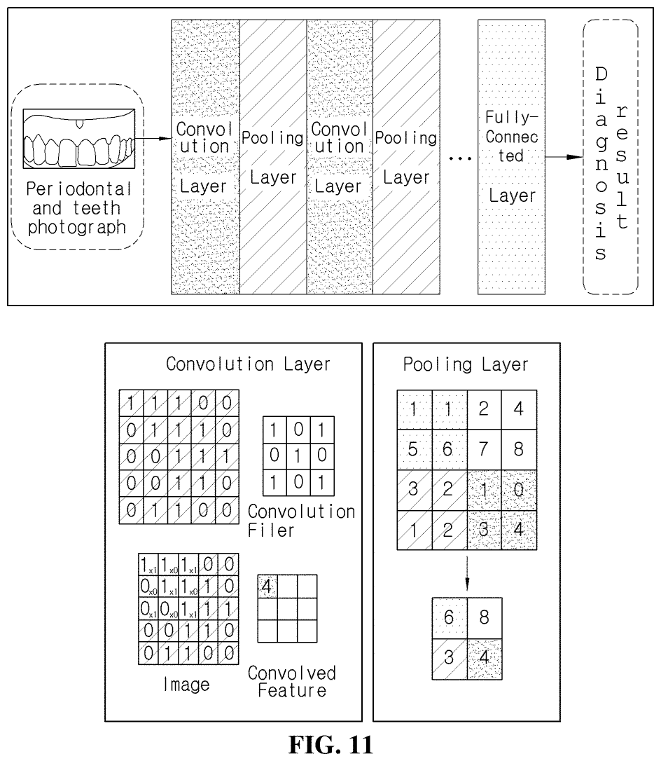

As shown in FIG. 11, the convolutional neural network (CNN) algorithm refers to an algorithm that finally outputs a diagnosis result (periodontal disease analysis data) through a fully-connected layer after passing through a number of convolution layer, a pooling Layer, a convolution layer, and a pooling layer when periodontal and tooth photographs are entered. It is preferable to use Python libraries such as Tensorflow and Keras and set the size, numbers and strides of the convolution/pooling filters suitable for periodontal sites and sizes thereof.

In FIG. 6, the left side refers to an example of outputting a diagnosis result (a risk status as the periodontal disease analysis data and an index) by learning entire periodontal images through the CNN algorithm, and the right side refers to an example of outputting a diagnosis result (a risk status as periodical disease analysis data and an index) by dividing the periodontal images into 8 sites and individually learning the periodontal images through the CNN algorithm.

When the periodontal disease analysis data is outputted from the periodontal images through the CNN algorithm, the report generation device 22 generates a periodontal disease report and transmits the periodontal disease report to the user terminal 10 (S50).

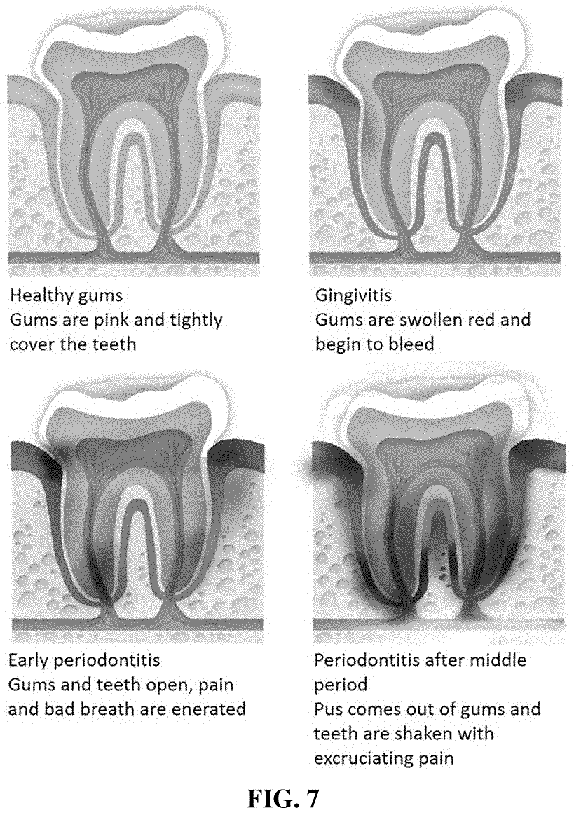

For example, the big data analysis unit 41 of the report generation device 22 extracts periodontal disease status information from the periodontal disease analysis data by using big data, which is the diagnosis and treatment data received from medical professionals in the existing dental general hospital. FIG. 7 is an example showing exacerbation stages of the periodontal disease. The periodontal disease analysis data is compared with the big data by using the example information on the periodontal disease exacerbation stages as big data, so that the periodontal disease status information may be extracted.

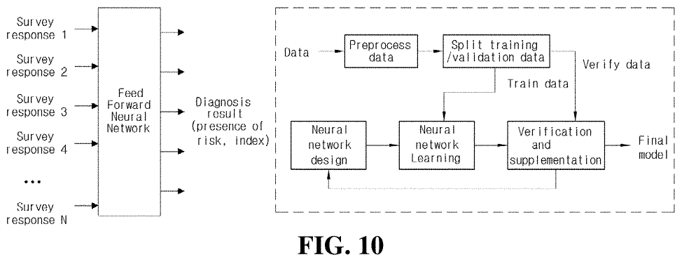

In addition, the inquiry classification and provision unit 42 classifies the inquiry information provided by the user through a feed forward neural network (FFNN) algorithm. In the FFNN algorithm, as shown in FIG. 10, the survey response (user information) is set as an input, and the diagnosis result is set as an output. The FFNN is designed suitably for an input/output structure using the Python-based libraries such as Tensorflow and Keras. The recently studied and analyzed FFNN algorithm is used for the activation function, loss function, optimization method, parallel computing structure, overfitting problem, and the like. The input data (survey response) is preprocessed, and the preprocessed data is divided into training data and verification data. Then, the learning data is trained through neural network learning, and the verification data and the learning result data are trained with a newly designed neural network through verification and supplementation procedures. In addition, when the verification and supplementation are completed, a final model is outputted as a survey diagnosis result.

Then, the report output and provision unit 43 generates the periodontal disease report by combining the periodontal disease status information analyzed by the big data analysis unit 41 and the survey diagnosis result obtained from the inquiry classification and provision unit 42. The periodontal disease report generated in the above manner is transmitted to the user terminal 10.

The periodontal disease report is provided together with a professional monitoring service for a regular periodontal care. The periodontal disease report is a report for a prevention, not for a medical practice conducted remotely or digitally. In other words, a periodontal disease status of the user is simply provided in the form of a report. When the user reads the provided periodontal disease report and determines that a visit to the hospital is necessary, the user requests a hospital reservation to the periodontal disease management server 20 through the user terminal 10 (S60). As the periodontal disease data is accumulated, personal teeth-related history is generated, and the user may track the provided status based on the generated report. When the hospital reservation is requested as needed, the periodontal disease management server 20 automatically reserves a hospital, and transmits hospital reservation information to the user terminal 10 (S70 and S80).

For the hospital search and reservation, a search and reservation for the optimal hospital is performed by any one or a combination of two or more of processes such as a search and reservation for a hospitals that can be booked quickly, a search and reservation for a hospital near the user's location, a search and reservation for a hospital by the self-ranking system, and a search and reservation for a hospital by name.

In other words, the hospitals to be reserved are searched based on location information, distance information between the user and the hospital, and available treatment time information, and the hospital is reserved according to the user's hospital selection in conjunction with the hospital server.

For example, based on a hospital name entered by the user or a self-recommended hospital and location information of hospitals around the user, a hospital having the shortest distance from the user's location is searched and recommended, and a hospital is automatically reserved in conjunction with a hospital server according to a hospital selection of the user.

Alternatively, a ranking may be determined by evaluating hospitals using consumer evaluations and an independent hospital evaluation algorithm, a reservation hospital may be searched based on the ranking, and a hospital may be reserved in conjunction with the hospital server according to a hospital selection the user.

According to the hospital reservation method using the ranking system, the evaluation is conducted using a consumer evaluation and an independent own hospital evaluation algorithm, and then ranked hospital information is extracted from a medical information database based on the evaluation result. Since desires of consumers (users) who want to receive higher medical services are necessary to be reflected due to the nature of the ranking system, it is preferable to include “hospitals available now for treatment” to provide the users more options.

According to the above-described present invention, just when the user photographs and uploads self periodontals, and fills out and sends an inquiry sheet, the periodontal status is analyzed through deep learning to provide the periodontal disease report to the user in real time, so as to the user to easily manage the periodontals, and prevent the periodontal disease from being exacerbated.

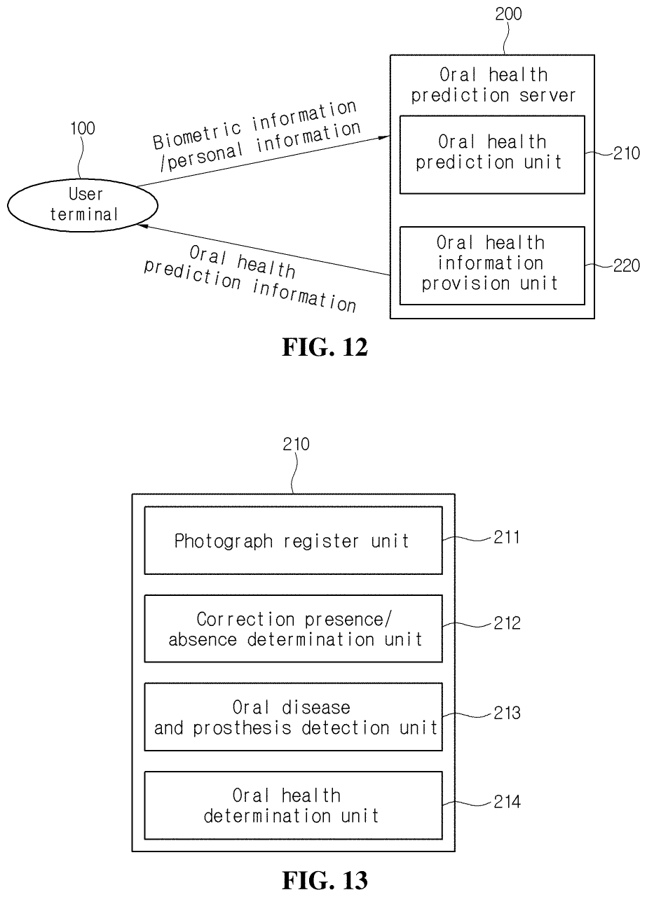

FIG. 12 is an entire configuration diagram of the oral health prediction apparatus using the machine learning algorithm according to a second preferred embodiment of the present invention, and the apparatus may include a user terminal 100 and an oral health prediction server 200.

The user terminal 100 and the oral health prediction server 200 may be connected to each other through various wired and wireless networks, and may be provided with communication interface in real time.

The user terminal 100 refers to a terminal used by the user trying to check an oral health status and serves to provide an oral image of the user and request an oral health determination result. The user may provide general data such as user personal information and inquiry data by using the user terminal 100.

It is preferable that the user receives the oral health status report through the user terminal 100, recognizes the own oral health status through the report, visits a dental hospital and takes follow-up measures to care the oral health when predicted that the oral health status is not good. The above user terminal 100 may be implemented with a mobile device such as a smartphone and a smart pad, and a personal computer and a notebook computer that are capable of accessing to Internet. In the present invention, it is assumed that the user terminal is implemented as a smart phone as the embodiment.

The oral health prediction server 200 serves to predict the oral health status by analyzing the oral image provided from the user terminal 100 through the machine learning algorithm (CNN, DNN, etc.). The above oral health prediction server 200 may analyze a dental caries status or periodontitis status, and a prosthesis status by analyzing the oral photographs, and predict the oral health status based on the analyzed dental caries status information, periodontitis status information, and prosthesis status information.

The oral health prediction server 200 may include an oral health prediction unit 210 that determines whether the photograph can be analyzed, whether a tooth is corrected, and whether a tooth is extracted by learning the oral image of the user through a convolutional neural network (CNN) algorithm, obtains the dental caries status information or the periodontitis status information and the prosthesis status information by performing an analysis through an object detection with respect to the determined result information, determines an oral health status by learning the obtained dental caries status information or periodontitis status information and prosthesis status information through an artificial neural network (ANN) algorithm, and an oral health information provision unit 220 configured to transmit the oral health status information determined by the oral health prediction unit 210 as oral health prediction information to the user terminal 100.

In addition, the oral health prediction unit 210 as shown in FIG. 13, may include a photograph register unit 211 for registering an oral photograph transmitted from the user terminal 100 as an oral health prediction target, a correction presence/absence determination unit 212 for determining whether the image can be analyzed, whether the image is corrected, and whether a teeth is extracted, by learning the image registered in the photograph register unit 211 through the convolutional neural network (CNN) algorithm, an oral disease and prosthesis detection unit 213 for obtaining dental caries status information or periodontitis status information and prosthesis status information by analyzing the result information, which is determined by the correction presence/absence determination unit 212, through the object detection, and an oral health determination unit 214 for determining the oral health status by learning the correction presence/absence information and tooth extraction information obtained from the correction presence/absence determination unit 212, and the dental caries status information or the periodontitis status information and the prosthesis status information obtained from the oral disease and prosthesis detection unit 213 through the artificial neural network (ANN) algorithm.

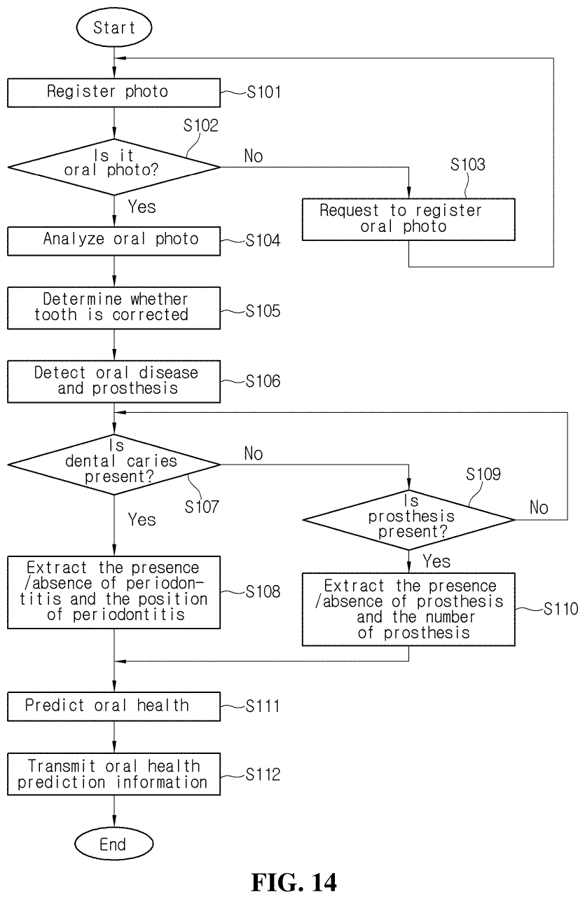

FIG. 14 is a flow chart of the first embodiment showing the oral health prediction method using the machine learning algorithm according to the present invention, in which the letter ‘S’ represents a step.

The oral health prediction method using the machine learning algorithm according to the present invention includes: (a) registering the oral image provided from the user terminal 100 as an oral health prediction target (S101), by an oral health prediction server 200 that predicts an oral health status by analyzing an oral image provided from a user terminal 100 through a machine learning algorithm, (b) determining, by the oral health prediction server 200, a presence or absence of an oral photograph by learning the oral image through a convolutional neural network (CNN) algorithm (S102 to S103), (c) determining whether the image is corrected and whether a tooth is extracted by learning the oral image through the convolutional neural network (CNN) algorithm, by the oral health prediction server 200 when the oral photograph is present (S104 to S105), (d) obtaining, by the oral health prediction server 200, dental caries status information and prosthesis status information by analyzing the determined correction status information and tooth extraction status information through the object detection (S106 to S110), and (e) determining, by the oral health prediction server 200, an oral health status by learning the correction status information, extraction status information, dental caries status information, and prosthesis status information by using an artificial neural network (ANN) algorithm (S111).

The dental caries status information may include information on the presence or absence of dental caries and information on the number of dental caries when the dental caries is present, and the prosthesis status information may include information on the presence or absence of a prosthesis and information on the number of prostheses when the prosthesis is present.

In addition, the first embodiment of the oral health prediction method using the machine learning algorithm according to the present invention may further include (f) transmitting the oral health prediction information obtained through the determination in step (e) to the user terminal 100 (S112).

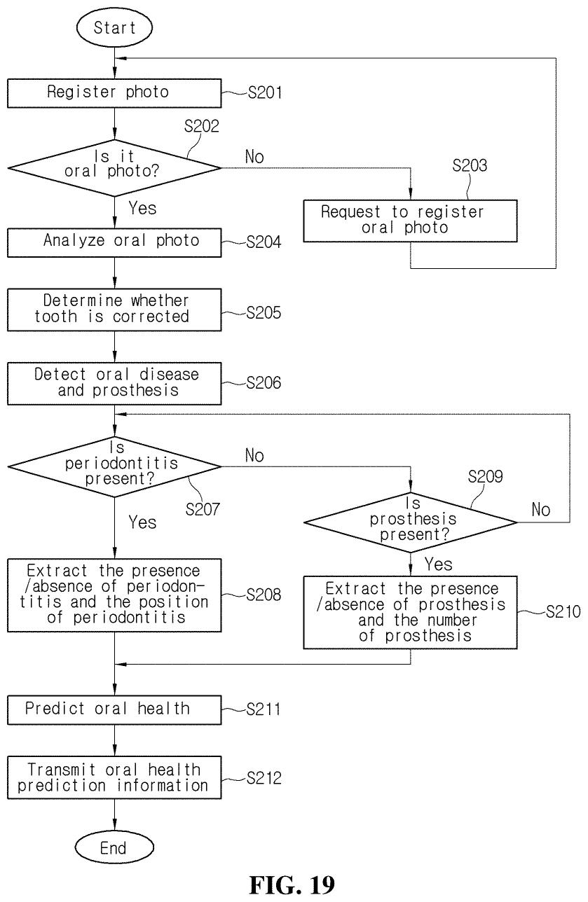

FIG. 19 is a flow chart of the second embodiment showing the oral health prediction method using the machine learning algorithm according to the present invention, in which the letter ‘S’ represents a step.

The second embodiment of the oral health prediction method using the machine learning algorithm according to the present invention (a) registering the oral image provided from the user terminal 100 as an oral health prediction target, by an oral health prediction server 200 that predicts an oral health status by analyzing an oral image provided from a user terminal 100 through a machine learning algorithm (S201), (b) determining, by the oral health prediction server 200, a presence or absence of an oral photograph by learning the oral image through a convolutional neural network (CNN) algorithm (S202 to S203), (c) determining whether the image is corrected and whether a tooth is extracted by learning the oral image through the convolutional neural network (CNN) algorithm, by the oral health prediction server 200 when the oral photograph is present (S204 to S205), (d) obtaining, by the oral health prediction server 200, periodontitis status information and prosthesis status information by analyzing the determined correction status information and tooth extraction status information through the object detection (S206 to S210), and (e) determining, by the oral health prediction server 200, an oral health status by learning the correction status information, tooth extraction status information, periodontitis status information, and prosthesis status information by using an artificial neural network (ANN) algorithm (S211).

The periodontitis status information may include periodontitis presence/absence information and position information of periodontitis when the periodontitis is present, and the prosthesis status information may include information on the presence or absence of a prosthesis and information on the number of prostheses when the prosthesis is present.

In addition, the second embodiment of the oral health prediction method using the machine learning algorithm according to the present invention may further include (f) transmitting the oral health prediction information obtained through the determination in step (e) to the user terminal 100 (S212).

The first embodiment of the oral health prediction method using the machine learning algorithm according to the present invention is a method of predicting an oral health in the case that the oral disease is dental caries, and the second embodiment of the oral health prediction method using the machine learning algorithm is a method of predicting an oral health in the case that the oral disease is periodontitis. The process of predicting overall oral health is the same. However, only classifications of the oral diseases, which are prerequisites for predicting the oral health, are different. Hereinafter, the first and second embodiments of the oral health prediction method using the machine learning algorithm according to the present invention will be described together for convenience of description.

The oral health prediction apparatus and method using a dental caries detection configured in the above manner according to the present invention will be described in detail as follows.

First, the user downloads an oral health prediction application for checking and caring an oral health condition from the oral health prediction server 200 and stores the oral health prediction application in the user terminal 100.

Subsequently, in order to check the oral health status, the user executes the oral health application, and provides oral photographs, personal information, and the like to request an oral health diagnosis. Inquiry data for the oral health diagnosis may be written and provided as necessary. The inquiry data refers to an inquiry sheet and may be provided in a check list form. The inquiry sheet may include items for checking whether a tooth is corrected.

The oral photograph is preferably a photograph of the entire oral cavity, and a photograph of only a part of the oral cavity may be used.

In the oral health prediction server 200, when the oral photograph, personal information and/or inquiry data are received through the user terminal 100 accessed through the network, the oral health prediction unit 210 learns oral photographs (images) through a convolutional neural network (CNN) algorithm to determine whether the photograph can be analyzed, whether the tooth is corrected, and whether the tooth is extracted, analyzes the determined result information through an object detection to obtain dental caries status information or periodontitis status information and prosthesis status information, and learns the obtained dental caries status information and prosthesis status information through an artificial neural network (ANN) algorithm to determine the oral health status.

For example, the photograph registration unit 211 of the oral health prediction unit 210 registers the oral photograph transmitted by the user as an oral health prediction target image in an internal database. Since users personally and directly photograph and photographing environments are different, there is no standard for colors of actual photographed images. Accordingly, a reference point may be automatically set with respect to the oral images, colors of all photographs may be corrected to correspond to the reference point, and then the corrected photographs may be saved (S101 and S201).

Next, the correction presence/absence determination unit 212 determines a presence or absence of an oral photograph by learning the oral image through a convolutional neural network (CNN) algorithm (S102 to S103). When the oral photograph is not present as a result of the determination, a text message, which informs that there is no oral photograph and the oral photograph is required to be registered, is transmitted to the user terminal 100 (S103 and S203).

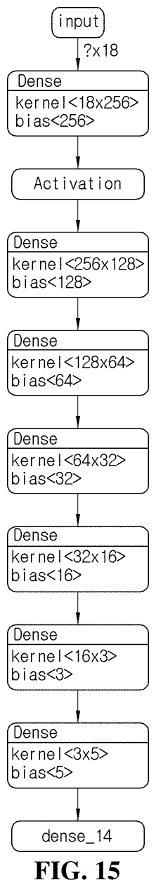

FIG. 15 shows a process of determining the presence or absence of the oral photograph by learning the oral image using the CNN algorithm. To this end, it is assumed that a CNN model, which is configured to determine whether the photograph taken and requested for the registration is an oral photograph, has been constructed in advance. As for the previously trained CNN model, an oral photograph category may be preferably added to a known CNN model such as VGG16 (https://neurohive.io/en/popular-networks/vgg16/) and ResNet50. The training data may include oral photograph data and photograph data of the category to be added.

The presence or absence of the oral photograph may be determined by learning through the CNN model by using the above oral photograph data. However, in this case, a lot of learning time may be required according to the amount of added training data.

Accordingly, as an alternative method of determining the presence or absence of the oral photograph, the process of determining the presence or absence of the oral photograph may be omitted by using the above oral health prediction application instead of using the CNN algorithm to induce the user not to register an image other than an oral photograph. A guidance text may be use as the method of inducing the user not to register an image other than the oral photograph.

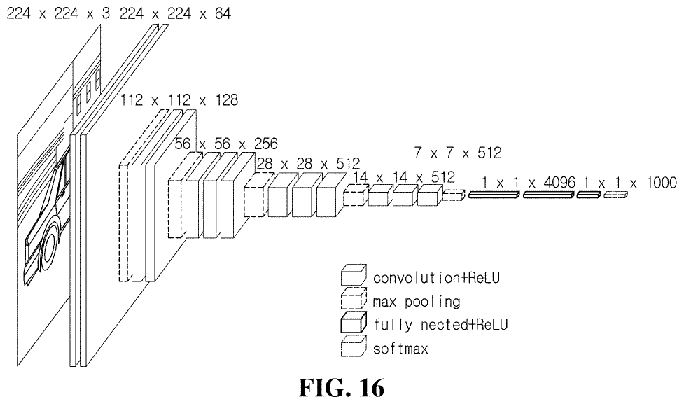

As a result of the confirmation of step S102 or S202, when the photograph is determined as an oral image, the correction presence/absence determination unit 212 subsequently determines the presence or absence of a correction by learning the oral image using the convolutional neural network (CNN) algorithm shown in FIG. 16 (S104 to S105 and S204 to S205). FIG. 16 is an example of the CNN model for determining the presence or absence of correction from the inputted oral image. The CNN model may be constructed using AutoKeras. A transfer learning using previously trained VGG16, ResNet50, or the like may also be used. The required data is oral photograph data with or without braces, and the presence/absence of correction may be simply determined by adding two categories of the oral photograph (with or without braces) to the CNN model.

Although the presence/absence of correction may be determined using the CNN model, sufficient learning time and data are also required, and an increase of learning data causes an increase in the amount of computation as a result. Accordingly, the learning process of the convolutional neural network (CNN) algorithm for determining the presence/absence of correction may be omitted by inducing the user to fill out the inquiry sheet to select the presence/absence of correction.

When inquiry information is provided after the inquiry sheet is filled out, the inquiry information is classified by the feed forward neural network (FFNN) algorithm. The FFNN algorithm refers to a machine learning algorithm in which a survey response (user information) is inputted and a diagnosis result is outputted. It is preferable to design and use the FFNN suitable for an input/output structure by using Python-based libraries such as Tensorflow and Keras. The recently studied and analyzed FFNN algorithm may be used for the activation function, loss function, optimization method, parallel computing structure, overfitting problem, and the like. The input data (survey response) is preprocessed, and the preprocessed data is divided into training data and verification data. Then, the learning data is trained through neural network learning, and the verification data and the learning result data are trained with a newly designed neural network through verification and supplementation procedures. In addition, when the verification and supplementation are completed, a final model is outputted as a survey diagnosis result.

The presence/absence of correction may be determined using the CNN model constructed herein, however, the presence/absence of tooth extraction may be additionally determined using the same CNN model. When a category of the tooth extraction oral photograph is added to the CNN model, the presence/absence of tooth extraction may be simply determined.

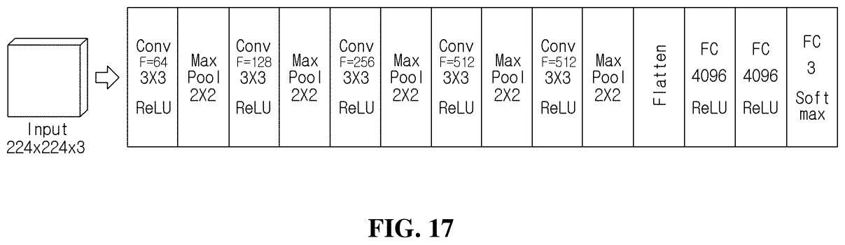

Next, the oral disease and prosthesis detection unit 213 obtains dental caries status information or periodontitis status information and prosthesis status information by analyzing the determined correction status information and tooth extraction status information through an object detection (S106 to S110 and S206 to S210). To this end, an object detection algorithm, which determines the detection of oral disease (dental caries and periodontitis) and prosthesis from the inputted section photograph (image) as shown in FIG. 17, is constructed. The object detection algorithm for determining the detection of oral disease and prosthesis may use previously developed Faster R-CNN, SSD, YOLO (W. Liu et al., 2015, arXiv (http://arxiv.org/abs/1512.02325)), and the like. Photograph data indicated with the oral disease or prosthesis may be used as necessary data for obtaining dental caries status information, periodontitis status information, and prosthesis status information through the object detection algorithm. An accuracy may be improved by separately learning according to whether the brace is worn. Accordingly, both of the dental caries or periodontitis and the prosthesis can be detected by using one algorithm. The dental caries status information may include information on the presence or absence of dental caries and information on the number of dental caries when the dental caries is present, and the prosthesis status information may include information on the presence or absence of a prosthesis and information on the number of prostheses when the prosthesis is present. In the dental caries image learning, an accurate site of the oral photograph provided by the user is checked in the entire oral site, a position of the oral site image is confirmed, and then the image learning is performed, thereby extracting the presence or absence of dental caries and the number of dental caries. In the same way, the presence or absence of prosthesis and the number of prosthesis are also extracted.

In addition, the periodontitis status information may include periodontitis presence/absence information and position information of periodontitis when the periodontitis is present, and the prosthesis status information may include information on the presence or absence of a prosthesis and information on the number of prostheses when the prosthesis is present. In the periodontitis image learning, an accurate site of the oral photograph provided by the user is checked in the entire oral site, a position of the oral site image is confirmed, and then the image learning is performed, thereby extracting the presence or absence of periodontitis and the number of periodontitis. For the analysis of periodontitis, the affected area of periodontitis is generally classified into 12 sites. Accordingly, the oral photograph (periodontal image) transmitted by the user is compared with the classified 12 sites to confirm an accurate position of the affected area so that the position of periodontitis is recognized. In the same way, the presence or absence of prosthesis and the number of prosthesis are also extracted.

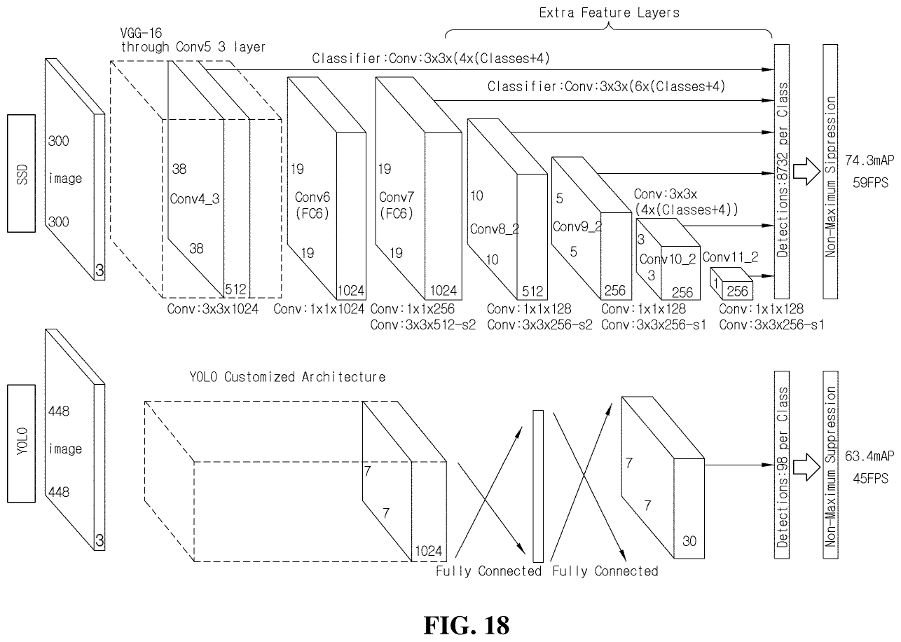

Then, the oral health determination unit 214 determines the oral health status by learning the correction status information, the extraction status information, the dental caries status information or periodontitis status information, and the prosthesis status information through the artificial neural network (ANN) algorithm as shown in FIG. 18 (a model proposed by industrial mathematics problem solving workshop of the NIMS in 2019). The ANN algorithm serving as a machine learning algorithm determines the oral health status (oral health level) by inputting the correction status information, the extraction status information, the dental caries status information or periodontitis status information, and the prosthesis status information. To this end, the patient's oral health level determination data, which corresponds to reliable photograph data obtained from a medical specialist, resident or intern, may be used. When the above oral health status determination is completed, oral health prediction information is generated and stored in an internal database.

Next, the oral health information provision unit 220 transmits the oral health prediction information obtained through the determination to the user terminal 100. The oral health prediction information may have a report format. To this end, the oral health information provision unit 220 may be provided with a communication module capable of transmitting and receiving data to and from the user terminal 100.

The oral health prediction information is provided together with a professional monitoring service for a regular oral care. The oral health prediction information is a report for a prevention, not for a medical practice conducted remotely or digitally. In other words, an oral health status of the user is simply provided in the form of a report. When the user reads the provided oral health report and determines that a visit to the hospital is necessary, it is preferable that the user visits a hospital and allows the oral problem to be treated so as to care the oral health.

According to the above-described present invention, when the user photographs and uploads an oral cavity, and fills out and sends an inquiry sheet, the oral disease and the prosthesis are detected by using the machine learning algorithm, the oral health status is predicted using the detection and the oral health report is provided to the user in real time, so that the user can easily care the individual oral health, and the oral health can be prevented from being exacerbated.

The present implemented by the inventor is described in detail according to the above embodiments, however, it would be appreciated by those skilled in the art that the present invention is not limited to the described embodiments and various modifications may be made to those embodiments without departing from the spirit of the invention.

### INDUSTRIAL APPLICABILITY

The present invention may be applied to a technology for analyzing an oral image to predict an oral health in an oral health prediction apparatus.

1. An apparatus for predicting an oral health by analyzing an oral photograph using a machine learning algorithm, the apparatus comprising:
a user terminal for providing a periodontal image, personal information, and inquiry data of a user, and requesting a periodontal disease report; and
a periodontal disease management server that analyzes the periodontal image provided from the user terminal by using a deep learning to generate a periodontal disease report and transmits the periodontal disease report to the user terminal.

2. The apparatus of claim 1, wherein the periodontal disease management server automatically searches for a hospital that responds to symptoms in the periodontal disease report upon request of a hospital reservation from the user terminal, and automatically makes a reservation in conjunction with a plurality of hospital servers.

3. The apparatus of claim 1, wherein the user terminal generates the periodontal image by photographing an affected area of the user using a camera, and transmits the generated periodontal image to the periodontal disease management server.

4. The apparatus of claim 1, wherein the periodontal disease management server includes an information analysis device for extracting analysis data after learning the periodontal image of the user by using the deep learning.

5. The apparatus of claim 4, wherein the information analysis device includes:
an image learning unit for learning the periodontal image;
an image analysis unit for analyzing a result learned by the image learning unit; and
an image diagnosis unit that extracts periodontal disease analysis data by analyzing the periodontal image through deep learning based on the image analysis result.

6. The apparatus of claim 4, wherein the periodontal disease management server further includes:
a report generation device that analyzes the analysis data extracted from the information analysis device and inquiry information based on big data to generate the periodontal disease report, and transmits the generated periodontal disease report to the user terminal.

7. The apparatus of claim 6, wherein the report generation device includes:
a big data analysis unit for analyzing periodontal disease analysis data provided by the information analysis device 21 based on periodontal disease big data;
an inquiry classification and provision unit for providing inquiry data to the user terminal, and classifying the inquiry information provided from the user terminal; and
a report output and provision unit for outputting the periodontal disease report based on the analysis result of the big data analysis unit and the inquiry classification information of the inquiry classification and provision unit, and providing the outputted periodontal disease report.

8. The apparatus of claim 4, wherein the periodontal disease management server further includes:
a hospital reservation device for searching for a hospital corresponding to symptoms of the periodontal disease report to automatically make a reservation when a hospital reservation is requested through the user terminal, and transmitting hospital reservation information to the user terminal.

9. The apparatus of claim 8, wherein the hospital reservation device searches and recommends a hospital having a distance shortest from a user location based on a hospital name entered by the user or a self-recommended hospital and location information of hospitals around the user, and automatically makes a hospital reservation in conjunction with a hospital server according to a hospital selection of the user.

10. The apparatus of claim 8, wherein the hospital reservation device determines a ranking by evaluating hospitals using consumer evaluations and a self hospital evaluation algorithm, searches for a reservation hospital based on the ranking, and makes a hospital reservation according to a hospital selection of the user in conjunction with a hospital server.

11. An apparatus for predicting an oral health by analyzing an oral photograph using a machine learning algorithm, the apparatus comprising:
a user terminal for providing an oral image of a user and requesting an oral health determination result; and
an oral health prediction server that predicts an oral health status by analyzing an oral image provided from the user terminal through a machine learning algorithm, wherein
the oral health prediction server analyzes a dental caries status or periodontitis status, and a prosthesis status by analyzing the oral photograph, and predicts the oral health status based on the analyzed periodontitis status information and prosthesis status information.

12. The apparatus of claim 11, wherein the oral health prediction server includes:
an oral health prediction unit that determines whether the photograph can be analyzed, whether a tooth is corrected, and whether a tooth is extracted by learning the oral image of the user through a convolutional neural network (CNN) algorithm, obtains dental caries status information or periodontitis status information and prosthesis status information by performing an analysis through an object detection with respect to the determined result information, and determines an oral health status by learning the obtained dental caries status information or periodontitis status information and prosthesis status information through an artificial neural network (ANN) algorithm.

13. The apparatus of claim 12, wherein the oral health prediction server further includes:
an oral health information provision unit configured to transmit the oral health status information determined by the oral health prediction unit as oral health prediction information to the user terminal.

14. The apparatus of claim 12, wherein the oral health prediction unit includes:
a correction presence/absence determination unit that determines whether the photograph can be analyzed, whether a tooth is corrected, and whether a tooth is extracted by learning a registered oral image through the CNN algorithm;
an oral disease and prosthesis detection unit for obtaining the dental caries status information or the periodontitis status information and the prosthesis status information by analyzing the result information, which is determined by the correction presence/absence determination unit, through the object detection; and
an oral health determination unit for determining the oral health status by learning the correction presence/absence information and the tooth extraction presence/absence information obtained from the correction presence/absence determination unit, and the dental caries status information or the periodontitis status information and the prosthesis status information obtained from the oral disease and prosthesis detection unit through the ANN algorithm.

15. An oral health prediction method using a machine learning algorithm with an apparatus for predicting an oral health by analyzing an oral photograph through the machine learning algorithm, the method comprising:
(a) registering an oral image provided from the user terminal as an oral health prediction target, by an oral health prediction server that predicts an oral health status by analyzing the oral image provided from a user terminal through a machine learning algorithm;
(b) determining, by the oral health prediction server, a presence or absence of the oral photograph by learning the oral image through a convolutional neural network (CNN) algorithm;
(c) determining, by the oral health prediction server upon a presence of the oral photograph, whether the image is corrected and whether a tooth is extracted by learning the oral image through the CNN algorithm;
(d) obtaining dental caries status information or periodontitis status information and prosthesis status information by analyzing correction status information and tooth extraction status information determined by the oral health prediction server through an object detection; and
(e) determining, by the oral health prediction server, an oral health status by learning the correction status information, the extraction status information, the dental caries status information, and the prosthesis status information through an artificial neural network (ANN) algorithm.

16. The method of claim 15, wherein the dental caries status information includes presence/absence information of dental caries and number information of the dental caries when the dental caries is present.

17. The method of claim 15, wherein the periodontitis status information includes presence/absence information of periodontitis and position information of the periodontitis when the periodontitis is present.

18. The method of claim 15, wherein the prosthesis status information includes presence/absence information of prosthesis and number information of the prostheses when the prosthesis is present.

19. The method of claim 15, further comprising:
(f) transmitting the oral health prediction information obtained through the determination in step (e) to the user terminal.

20. The method of claim 15, wherein a step of providing guidance information for inducing the user not to register an image other than an oral photograph through an oral health prediction application is replaced instead of step (b), thereby omitting a learning process of the CNN algorithm for determining whether the oral photograph is present.
